(19) United States
(12) Patent Application Publication
(10) Pub. No.: US 2005/0014834 A1
(43) Pub. Date: Jan. 20, 2005

(54) Selective anesthetic agents and methods of identifying the same

(76) Inventors: David Stuart Reynolds, Bishops Stortford (GB); Thomas Walter Rosahl, Hoddesdon (GB); Keith Alan Wafford, Much Hadham (GB); Paul John Whiting, Stansted Mountfitchet (GB)

Correspondence Address: MERCK AND CO INC, P O BOX 2000, RAHWAY, NJ, 070650907, US

(21) Appl. No.: 10/498,257

(22) PCT Filed: Dec. 11, 2002
(86) PCT No.: PCT/GB02/05619

(30) Foreign Application Priority Data
Dec. 12, 2001 (GB) 0129709.2
Oct. 24, 2002 (GB) 0224787.2

(51) Int. Cl. A61K 31/195

(52) U.S. Cl. 514/561

(57) ABSTRACT

The present invention relates to the use of GABA_{A} receptor β3 selective anesthetic agents for the manufacture of medicaments for providing anesthesia; to transgenic animals of use in the testing of such agents; and to methods for screening for GABA_{A} receptor β3 selective anesthetic agents.

### BACKGROUND OF THE INVENTION

The present invention relates to the use of a class of anesthetics. More particularly, this invention is concerned with the use of anesthetics which are ligands for γ-aminobutyric acid type A (GABA_{A}) receptors. In particular, this invention is concerned with the use of anesthetics that have a selective action at GABA_{A} receptors containing a β₃ subunit. The present invention also provides a transgenic non-human animal and uses thereof.

Receptors for the major inhibitory neurotransmitter, gamma-aminobutyric acid (GABA), are divided into two main classes: (1) GABA_{A} receptors, which are members of the ligand-gated ion channel superfamily; and (2) GABA_{B} receptors, which are members of the G-protein coupled receptor superfamily. Since the first cDNAs encoding individual GABA_{A} receptor subunits were cloned the number of known members of the mammalian family has grown to include at least six α subunits, three β subunits, three γ subunits, one δ subunit, one θ, and one ε subunit. The native GABA_{A} receptor is a pentameric assembly of subunits drawn from these classes. It has been indicated that an α subunit, a β subunit and a γ subunit constitute the minimum requirement for forming a fully functional GABA_{A} receptor expressed by transiently transfecting cDNAs into cells. As indicated above, a θ, δ and ε subunit also exist, but these are present only to a minor extent, in GABA_{A} receptor populations.

It is now generally recognised that GABA_{A} receptors are an important anesthetic target. At anesthetic or subanesthetic concentrations many structurally diverse general anesthetics act to potentiate GABA_{A} receptor-mediated neuronal responses. Higher concentrations of such agents can elicit direct activation of the receptor in the absence of GABA—see, for instance, D. L. Tanelian et al., Anesthesiology, 78(4), 757-776 (1993); N. P. Franks and W. R. Lieb, Nature, 367, 607-614 (1994); and R. A. Harris et al., FASEB J., 9, 1454-1462 1995).

Potentiation of the action of GABA by most anesthetics shows little in the way of subunit specificity. Thus, for instance, with both native and all recombinant GABA_{A} receptors studied to date, both alphaxalone and propofol markedly potentiate the action of GABA. In contrast, the direct action of etomidate at GABA_{A} receptors is influenced by the type of β subunit. The potentiation and direct activation of receptors by etomidate favours receptors containing either a β₂ or a β₃ subunit, versus a β₁ subunit—see also C. Hill-Venning et al., Br. J. Pharmacol., 120, 749-756 (1997).

This selectivity of etomidate for β₂- or β₃-containing receptors is determined by a single amino acid. The mutation of asparagine at position 289 (which is present within the channel domain of the β₃ subunit) to serine (the homologous residue in β₁) strongly suppressed the GABA-modulatory and GABA-mimetic effects of etomidate—see D. Belelli et al., Proc. Natl. Acad. Sci. USA, 94, 11031-11036 (1997).

### SUMMARY OF THE INVENTION

We have now found that a GABA_{A} receptor β₃ selective anesthetic agent will have the highly desirable characteristic of affording rapid recovery. A genetically engineered “knock-in” mouse that has the β₂ subunit mutated at the single key amino acid (β2N289S) has been prepared such that etomidate will only be acting on the mice through GABA_{A} receptors containing β₃ subunits. When treated with anesthetic doses of etomidate, these β2N289S mice show a dramatically faster recovery of performance in the rotarod assay, compared with similarly treated wildtype mice.

### BRIEF DESCRIPTION OF FIGURES

FIG. 1 shows the targeting strategy for the GABA_{A} receptor β2N289S knock-in mice. (a) Schematic representation of the wildtype β2 allele of the GABA_{A} receptor and (b) targeting vector including the site-specific β2N289S mutation indicated by an asterisk. E, EcoRI; K, KpnI restriction sites; 6, 7 and 8, exons 6, 7 and 8, respectively; black arrowhead, loxP site; neo, neomycin resistance gene; TK, thymidine kinase gene. (c) Targeted β2 allele after homologous recombination before and (d) after cre-mediated excision of the loxP flanked neomycin cassette.

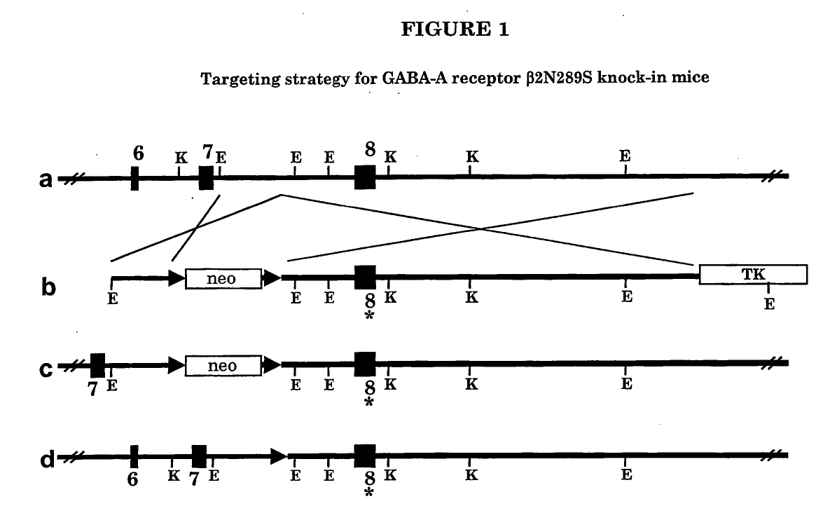

FIG. 2 shows the anesthetic effects of etomidate in β2N289S knock-in mice measured by the loss of the righting reflex.

FIG. 3 shows the recovery of wildtype and β2N289S knock-in mice following etomidate-induced anesthesia.

FIG. 4 shows the level of anesthesia induced by etomidate as determined by electroencephalography (EEG) in wildtype and β2N289S knock-in mice.

FIG. 5 shows the level of “hypnotic hangover” measured as an effect on slow wave sleep following recovery from etomidate anesthesia in wildtype and β2N289S knock-in mice.

### DETAILED DESCRIPTION OF THE INVENTION

Thus, according to a first aspect of the present invention, there is provided the use of a GABA_{A} receptor β₃ selective anesthetic agent for the manufacture of a medicament for providing anesthesia.

There is further provided a method for providing anesthesia, comprising administering to a mammal a GABA_{A} receptor β₃ selective anesthetic agent in an amount equal to or in excess of an amount effective for anesthesia in order to induce or maintain anesthesia.

Reference herein to the “GABA_{A} receptor” will be understood to be, most preferably, a reference to the human GABA_{A} receptor.

As used herein, the term “GABA_{A} receptor β₃ selective anesthetic agent” refers to an anesthetic agent which has a selective modulatory action at GABA_{A} receptors containing a β₃ subunit. Thus, compounds of use in the present invention will produce a potent enhancement of the control response evoked by GABA on a GABA_{A} receptor containing a β₃ subunit. Such compounds will ideally have an EC₅₀ of ≦10 μM, and preferably ≦5 μM. The compounds of use in this invention will possess at least a 2-fold, suitably at least a 5-fold, and advantageously at least a 10-fold, selective affinity for the β₃ subunit, relative to the β₂ subunit. The compounds of use in this invention will also preferably show such selectivity relative to the β₁ subunit.

The compounds of use in the present invention may also possess an agonist action at the GABA_{A} receptor containing a β₃ subunit, in the absence of GABA. Such compounds will ideally have an EC₅₀ of ≦25 μM, and preferably ≦10 μM. The compounds of use in this invention will possess at least a 2-fold, suitably at least a 5-fold, and advantageously at least a 10-fold, selective agonist activity at the β₃ subunit, relative to the β₂ subunit. The compounds of use in this invention will also preferably show such selectivity relative to the β₁ subunit.

Therefore, according to another aspect of the present invention, there is provided the use of GABA_{A} receptor β₃ selective anesthetic agent for the manufacture of a medicament for providing anesthesia, wherein said anesthetic agent is a modulator of the GABA_{A} receptor containing a β₃ subunit, having potency (EC₅₀) for such a receptor of 10 μM or less, which elicits at least a 30% potentiation of the GABA EC₂₀ response in transfected L(tk−) cells expressing the GABA_{A} receptor containing the β₃ subunit, and which elicits at most a 20% potentiation of the GABA EC₁₀ response in transfected L(tk−) cells expressing the GABA_{A} receptor containing the β₂ subunit.

This aspect of the present invention also provides a method for providing anesthesia comprising administering to a mammal a GABA_{A} receptor β₃ functionally selective anesthetic agent in an amount equal to or in excess of an amount effective for anesthesia in order to induce or maintain anesthesia, wherein said anesthetic agent is a modulator of GABA_{A} receptor containing a β₃ subunit, having potency (EC₅₀) for such a receptor of 10 μM or less, which elicits at least a 30% potentiation of the GABA EC₂₀ response in transfected L(tk−) cells expressing the GABA_{A} receptor containing the β₃ subunit, and which elicits at most a 20% potentiation of the GABA EC₁₀ response in transfected L(tk−) cells expressing the GABA_{A} receptor containing the β₂ subunit.

In this aspect of the present invention, the potentiation of the GABA EC₂₀ response in L(tk−) cells expressing either the β₃ or β₂ subunits of the human GABA_{A} receptor can conveniently be measured by procedures readily available to the skilled worker, for instance, as described in C. Hill-Venning et al., Br. J. Pharmacol., 120, 749-756 (1997); McKernan et al., Nat. Neurosci., 3, 587-592 (2000).

The compounds of use in this aspect of the invention will elicit at least a 30%, preferably at least a 40%, and more preferably at least a 50%, potentiation of the GABA EC₂₀ response in transfected L(tk−) cells expressing the GABA_{A} receptor containing the β₃ subunit. Moreover, the compounds of use in this aspect of the invention will elicit at most a 20%, preferably at most a 10%, and more preferably 0%, potentiation of the GABA EC₂₀ response in transfected L(tk−) cells expressing the GABA_{A} receptor containing the β₂ subunit.

According to another aspect of the present invention, there is provided the use of GABA_{A} receptor β₃ functionally selective anesthetic agent for the manufacture of a medicament for providing anesthesia, wherein said anesthetic agent is an agonist at the GABA_{A} receptor containing a β₃ subunit, having potency (EC₅₀) for such a receptor of 25 μM or less, when measured using a whole-cell clamp technique on rodent fibroblast L(tk⁻) cells stably transfected with the GABA_{A} receptor containing the β₃ subunit, and which has a potency (EC₅₀) for the GABA_{A} receptor containing a β₂ subunit of at most 50 μM, when measured using a whole-cell clamp technique on rodent fibroblast L(tk⁻) cells stably transfected with the GABA_{A} receptor containing the β₂ subunit.

This aspect of the present invention also provides a method for providing anesthesia comprising administering to a mammal a GABA_{A} receptor β₃ functionally selective anesthetic agent in an amount equal to or in excess of an amount effective for anesthesia in order to induce or maintain anesthesia, wherein said anesthetic agent is an agonist at the GABA_{A} receptor containing a β₃ subunit, having potency (EC₅₀) for such a receptor of 25 μM or less, when measured using a whole-cell patch clamp technique on rodent fibroblast L(tk⁻) cells stably transfected with the GABA_{A} receptor containing the β₃ subunit, and which has a potency (EC₅₀) for the GABA_{A} receptor containing a β₂ subunit of at most 50 μM, when measured using a whole-cell clamp technique on rodent fibroblast L(tk⁻) cells stably transfected with the GABA_{A} receptor containing the β₂ subunit.

In this aspect of the present invention, the GABA-mimetic action of the compounds in stably transfected rodent fibroblast L(tk⁻) cells expressing either the β₃ or β₂ subunits of the human GABA receptor can conveniently be measured by procedures readily available to the skilled worker, for instance, as described in McKernan et al., Nat. Neurosci., 3, 587-592 (2000); D. Belelli et al., Proc. Natl. Acad. Sci., USA, 94, 11031-11036 (1997).

In order to elicit their behavioural effects, the compounds of use in the present invention will be brain-penetrant; in other words, these compounds will be capable of crossing the so-called “blood-brain barrier”. Preferably, the compounds of use in this aspect of the invention will be capable of exerting their beneficial therapeutic action following administration by the parenteral route.

For therapeutic application, pharmaceutical compositions may be provided which comprise one or more compounds of use in this invention in association with a pharmaceutically acceptable carrier. Preferably these compositions adapted for parenteral administration.

In particular, pharmaceutical intravenous formulations of a compound of use in the present invention will comprise one or more pharmaceutically acceptable excipients. Suitable liquid inert excipients/carriers include Water for Injection (USP in the US) and saline solution.

Other suitable excipients and other accessory additives include solvents, for example, ethanol, glycerol and propylene glycol; stabilizers, for example, EDTA (ethyl diamine tetraacetic acid) and citric acid; antimicrobial preservatives, for example, benzyl alcohol, methyl paraben and propyl paraben; buffering agents, for example, citric acid/sodium citrate, potassium hydrogen tartrate, sodium hydrogen tartrate, acetic acid/sodium acetate, maleic acid/sodium maleate, sodium hydrogen phthalate, phosphoric acid/potassium dihydrogen phosphate and phosphoric acid/disodium hydrogen phosphate; and tonicity modifiers, for example, sodium chloride, mannitol and dextrose.

It will be appreciated that the compounds of the present invention may be administered either alone or, more commonly, in combination with one or more agents. Such agents include intravenous anesthetics and inhaled anesthetics, for example, halothane, enflurane, isoflurane, methoxyflurane and nitrous oxide. The requirements for general anesthesia and surgery may necessitate the administration of several intravenous agents with different actions to ensure hypnosis, analgesia, relaxation and control of visceral reflex responses. Suitable intravenous anesthetics include barbiturates, for example, thiopental, methohexital and thiamylal; benzodiazepines, for example, diazepam, lorazepam and midazolam; opioid analgesics, for example, morphine, meperidine, fentanyl, sufentanil and alfentanil; neuroleptic-opioid combinations, for example, droperidol combined with an opioid analgesic such as fentanyl; ketamine; and propofol.

The novel effect of a GABA_{A} receptor β₃ selective anesthetic agent according to the present invention may be demonstrated using a transgenic animal, especially a transgenic mouse. Studies using recombinant receptors expressed in cell lines, have shown that substituting asparagine (Asn) for serine (Ser) at position 289 of the human GABA_{A} receptor β₂ subunit renders the resulting “mutant” receptors insensitive to etomidate. Furthermore, this point mutation does not affect the opening of the channel by GABA or the agonist actions of other classes of anesthetics such as barbiturates. The effect of GABA_{A} receptor β₃ functionally selective anesthetic agents according to the present invention may be demonstrated using β2N289S point mutation (“knock-in”) mice.

Thus, a further aspect of the present invention is based on a new transgenic animal that contains a GABA_{A} receptor β₂ subunit gene in which a portion of the open reading frame which encodes a region determining the selectivity of etomidate on the β₂ subunit polypeptide has been replaced with a corresponding portion of the GABA_{A} receptor β₁ subunit open reading frame. This animal is affected by anesthetic agents such as etomidate solely by virtue of the action of the drug on GABA_{A} receptors containing the β₃ subunit. The absence of the β₂ subunit correlates with a dramatically improved recovery from the effects of an anesthetic agent such as etomidate. Thus, the transgenic animals of the invention offer a unique animal model in which the genetic and biochemical mechanisms responsible for slow recovery from the effects of anesthetic agents such as etomidate can be investigated.

Accordingly, the invention features a transgenic animal (e.g., a non-human mammal, rodent, rat, mouse, rabbit, pig, cow, chicken, or fish) whose genomic DNA includes a gene having a GABA_{A} receptor β₂ subunit promoter operably linked to a DNA sequence encoding a point mutated GABA_{A} receptor β₂ subunit polypeptide. By “operably linked” is meant that a nucleotide sequence (e.g., an open reading frame) is linked to a regulatory sequence (e.g., a promoter) in a manner which allows for expression of the nucleotide sequence in vitro or in vivo. The transgenic animal exhibits rapid recovery from the effects of an anesthetic agent such as etomidate, as compared to a reference animal (e.g., the wildtype animal from which the transgenic animal was generated) whose genomic DNA does not contain the gene.

A “GABA_{A} receptor β₂ subunit promoter” refers to a promoter that directs transcription of an RNA in a temporal and cell-type specific manner at least substantially similar, if not identical, to the expression of a wildtype GABA_{A} receptor β₂ subunit gene. Thus, a “GABA_{A} receptor β₂ subunit promoter” includes wildtype GABA_{A} receptor β₂ subunit promoters as well as such promoters containing insertions, deletions, or substitutions that do not affect the temporal and tissue-specific expression of the GABA_{A} receptor β₂ subunit promoter. Similarly, a “point mutated GABA_{A} receptor β₂ subunit polypeptide” is a polypeptide that has at least one biochemical or cellular activity associated with a wildtype GABA_{A} receptor β₂ subunit polypeptide, and at least one (and preferably one) point mutation in the region determining etomidate sensitivity e of the β₂ subunit.

In addition, when the gene is made by replacing sequence encoding the region determining etomidate sensitivity, of a wildtype endogenous GABA_{A} receptor β₂ subunit gene with a corresponding GABA_{A} receptor β₁ subunit open reading frame, the transgenic animal can be homozygous or heterozygous for the mutated GABA_{A} receptor β₂ subunit gene. The invention also features a method of producing a transgenic animal of the invention by replacing at least a portion of a GABA_{A} receptor β₂ subunit gene in the genomic DNA of the transgenic animal with a DNA insert comprising a first sequence encoding a point mutated GABA_{A} receptor β₂ subunit polypeptide and a second sequence encoding a selectable marker; and removing the second sequence from the genomic DNA, thereby producing the transgenic animal.

In addition, the invention includes a nucleic acid comprising a GABA_{A} receptor β₂ subunit promoter operably linked to a sequence encoding a point mutated GABA_{A} receptor β₂ subunit polypeptide, which is useful for producing a transgenic animal of the invention.

The transgenic animals of the invention can be used to elucidate the biochemical and genetic determinants that provide for the mechanism of action of a β-2/3 selective anesthetic agent such as etomidate.

Suitable β₃ selective anesthetic agents for use in accordance with the present invention may be selected using methods that are readily apparent to a person of ordinary skill in the art. Thus, for instance, one such method for screening for β₃ selective anesthetic agents comprises:
a) exposing cells expressing a functional GABA_{A} receptor comprising the β₃ subunit (to the exclusion of any other β subunit) to putative anesthetic agents which may potentiate the effects of GABA at said GABA_{A} receptor;
b) selecting those agents that potentiate the effect of GABA at the receptor in step (a);
c) comparing the binding of the selected agents to the receptor in step (a) with the effects of the selected agents to other GABA_{A} receptors; and
d) determining those agents that potentiate the effect of GABA at the receptor in step (a) but with lower effects at other GABA_{A} receptors.

It will be appreciated that of the other possible GABA_{A} receptors referred to in steps (c) and (d), a functional GABA_{A} receptor comprising the β₂ subunit (to the exclusion of any other β subunit) is particularly preferred.

In addition, of the other possible GABA_{A} receptors referred to in steps (c) and (d), a functional GABA_{A} receptor comprising the β₁ subunit (to the exclusion of any other β subunit) is also preferred.

Stably co-transfected cell lines expressing suitable combinations of the GABA_{A} subunits are well known in the art. Thus, for example, stably co-transfected eukaryotic cell lines capable of expressing human GABA_{A} receptors comprising at least one α, at least one β and at least one γ subunit are described in International (PCT) Patent Publication Nos. WO 92/22652 and WO 94/13799, the contents of which are incorporated herein by reference. Stably co-transfected eukaryotic cell lines capable of expressing suitable combinations of the GABA_{A} subunits are also described in International (PCT) Patent Publication Nos. WO 96/10637, WO 98/23742 and WO 98/49293, the contents of which are incorporated herein by reference.

Other features or advantages of the present invention will be apparent from the following detailed description and also from the claims.

Introduction of a transgene into the fertilized egg of an animal (e.g., a mammal) is accomplished by any number of standard techniques in transgenic technology. See, for example, Hogan et al., Manipulating the Mouse Embryo: A Laboratory Manual, Cold Spring Harbor Laboratory Press, Cold Spring Harbor, N.Y., 1986. Most commonly, the transgene is introduced into the embryo by way of microinjection.

Once the transgene is introduced into the egg, the egg is incubated for a short period of time and is then transferred into a pseudopregnant animal of the same species from which the egg was obtained (Hogan et al., supra). In the case of mammals, typically 125 eggs are injected per experiment, approximately two-thirds of which will survive the procedure. Twenty viable eggs are transferred into pseudopregnant mammal, four to ten of which will develop into live progeny. Typically, 10-30% of the progeny (in the case of mice) carry the transgene.

To identify the transgenic animals of the invention, progeny are examined for the presence of the transgene using standard procedures such as Southern blot hybridization or PCR. Expression of the transgene can also be assessed using Northern blots, Western blots, and immunological assays.

In an alternative method the transgene is introduced via the homologous recombination technique into the mouse genome as outlined in Example 1 in more detail. In brief, genomic DNA containing the gene of interest is isolated from a genomic DNA library, sub-cloned into plasmid vectors and a detailed restriction endonuclease map is established. A gene targeting vector containing a long and a short region of homology to the gene of interest, as well as a positive and a negative selection marker, is introduced into embryonic stem (ES) cells. The frequency of homologous recombination upon positive-negative selection is unpredictable, but ranges typically from 1-10%. Correctly targeted ES cell clones are injected into blastocysts and the resulting chimeras test bred for germ line transmission. Progeny are tested by Southern blot hybridization or PCR analysis for the correct integration of the transgene and a colony of homozygous and wild-type control animals is established.

The intravenous anesthetic etomidate is known to mediate its effects through GABA_{A} receptors, and in particular by its action on β₂- and β₃-containing receptors. This selectivity is determined by a single amino acid, asparagine at position 289. In the β₁ subunit, which has reduced sensitivity to etomidate, there is a serine instead of an asparagine. In a preferred aspect of the present invention, there is provided a transgenic animal with a point mutation (serine for asparagine) at position 289 of the β₂ subunit. Thus, in particular, there is provided β2N289S point mutation (knock-in) mice.

Without further elaboration, it is believed that one skilled in the art can, based on the above disclosure and the description below, utilize the present invention to its fullest extent. The following examples are to be construed as merely illustrative of how one skilled in the art can practice the invention and are not limitative of the remainder of the disclosure in any way. All publications cited in this disclosure are hereby incorporated by reference.

### EXAMPLE 1

Generation of GABA_{A} Receptor β2N289S Knock-In Mice

The generation of mice carrying the codon change from Asn 289 to Ser 289 in the β₂ subunit of the GABA_{A} receptor in their genome was performed as follows. A murine 129/SvEv λ fixII library (Stratagene, LaJolla, Calif.) was screened using a PCR (polymerase chain reaction) product as a probe, which was amplified by using the following oligonucleotides: sense: 5′-TCT.CCA.TTC.TAG.ATT.ATA.AAC.TCA.TCA.CC-3′; antisense: 5′-GGT.CCA.TCT.TGT.CGA.CAT.CCA.GGC-3′. Two positive hybridising λ clones containing were subcloned via the NotI sites into pBluescript, one of them (A.1.2) containing ca. 17.5 kb and the other (J.1.1) 11 kb of genomic DNA. These two clones were further subcloned, an extensive DNA restriction map was established and the full 17.5 kb long DNA sequence, which contained exons 6, 7 and 8 of the β2 subunit gene, was determined. A 2 kb ERV subclone of JPI-H containing exon 8 was used for the site-directed mutagenesis. Single stranded DNA was prepared and the Asp 289 to Ser 289 codon change labelled by the novel restriction endonuclease restriction site ScaI which was introduced by standard techniques for site directed mutagenesis using oligonucleotide 5′-ACC.ACA.ATC.AGT.ACT.CAC.CTC.CGG.G-3′. The 2 kb ERV clone containing the β2N289S mutation was cloned into a 6 kb long SalI-ERV subclone (AE1-B) of full length clone A.1.2. The 8 kb long SalI DNA fragment of this newly generated clone (clone 8) was introduced into the SalI site and the 2 kb ERV+ERI DNA fragment of subclone JH3.3 blunt ended into the HpaI site of the pBS246-neo-tk-1 multiple utility targeting vector. pBS246-neo-tk-1 consists of the loxP site containing plasmid pBS246 (Sauer, Methods in Enzymology, 225, 890 (1993)) in which the phosphoglycerate kinase I (PGK) neo and the Herpes simplex virus thymidine kinase (TK) gene have been cloned. After linearization with NotI the targeting vector was introduced into AB2.2 ES cells (Lexicon Genetics Inc.) as described, for instance, by Thomas and Capecchi, Cell, 51, 503 (1987) and Rosahl et al., Cell, 75, 661 (1993). Homologous recombinants were identified by PCR using the oligonucleotides 5′-CTATGATGCCTCTGCTGCACGGGTTGC-3′ and 5′-GGATGCGGTGGGCTCTATGGCTTCTGA-3′ and were further confirmed by genomic Southern blotting. Presence of the β2N289S mutation was confirmed by cutting the 800 bp long PCR product amplified by primers 5′-TGGAAATTTGAGCAGCCCATTGTG-3′ and 5′-AACCTTTCATCTTTAGTCCCTGG-3′ with the restriction endonuclease ScaI. Correctly targeted ES cell clones were injected into C57BL6 blastocysts and one clone gave rise to highly chimeric males, which transmitted the targeted allele into the germ line. β2N289S+/−(heterozygous) mice were further bred with a cre-transgenic mouse (Schwenk et al., Nucleic Acid Res., 23, 5080 (1995)) to remove the neomycin resistance gene in their offspring. cre-Mediated recombination was confirmed by PCR using oligonucleotides 5′-AGATCTAGGTGATGACTGTC-3′ and 5′-AGACAGACGCCACATCACAC-3′. By further breeding, a colony of wildtype and homozygous β2N289S mice were generated and kept in a mixed 75% C57BL6/25% 129SvEv genetic background.

### EXAMPLE 2

Anesthetic Effects of Etomidate in β2N289S Knock-In and Wildtype Mice

Method

Loss of righting reflex (LORR) was used as a crude behavioural measure of anesthesia. Mice (n=11 wildtype, n=10 knock-in for each dose) were dosed intraperitoneally (i.p.) with 20, 30 or 40 mg/kg etomidate. Each mouse was placed in a separate cage and tested for LORR by gently rolling the mouse on it back. When the righting reflex had disappeared (usually around 90 seconds) the mouse was laid on it back on a piece of corrugated plastic and the time until righting reflex was regained was recorded. In order to avoid excessive heat loss during anesthesia a heat lamp was placed above the mice. To control for non-specific changes in anesthetic sensitivity induced by the mutation two different classes of anesthetics were also tested in this model: pentobarbitone, 50 mg/kg i.p. (n=11) and midazolam, 80 mg/kg i.p. (n=5).

Results

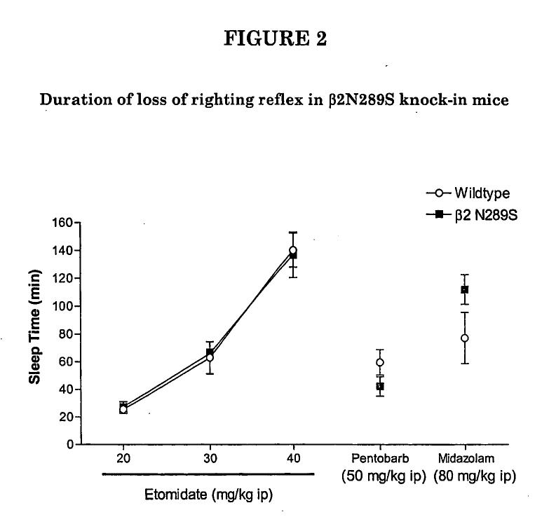

The duration of LORR in knock-in mice was nearly significantly similar to the duration seen in wildtype mice (FIG. 2; F_{1,57}=0.005, p=0.9498). Likewise, there was no significant difference between knock-in and wildtype mice for pentobarbitone or midazolam treatment (F_{1,28}=0.61, p=0.44). These data suggest that the anesthetic properties are mediated by the β₃ subunit and not by the β₂ subunit.

### EXAMPLE 3

Recovery of β2N289S and Wildtype Mice Following Etomidate Anesthesia

Method

Wildtype (n=30) and knock-in (n=30) mice were trained to perform the rotarod test of motor co-ordination. Mice were placed on a rod revolving at 16 rpm and the time taken for them to fall off recorded. Each mouse was given successive training trials until it could remain on the rod for 3 consecutive trials of 120 second duration. Mice were then split into 3 groups (n=10 for each genotype) and dosed i.p. with 15, 20 or 30 mg/kg etomidate. Mice were then placed back on the rotarod every 10 minutes (for 90 minutes), starting from the mean time for recovery of righting reflex for a given dose (obtained from experiment 1). In this way the rate of recovery of the ability to perform a motor task could be assessed.

Results

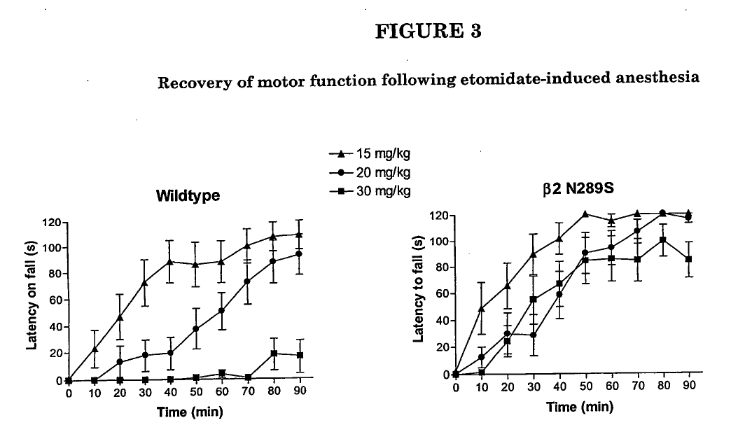

Following 15 mg/kg etomidate both wildtype and knock-in mice recovered rotarod performance at a similar rate, such that both groups could stay on for approximately 120 seconds 90 minutes after testing began. However, as the dose of etomidate increased to 20 mg/kg and especially 30 mg/kg, wildtype mice recovered more slowly than knock-in mice. FIG. 3 shows a clear effect on the rate of recovery with increasing dose of etomidate in the wildtype mice (F_{18,243}=5.80, p<0.00005). In contrast, the knock-in mice show a similar recovery rate across the dose range tested (F_{18,243}=1.45, p=0.111). These data suggest that β₂ is the primary mediator of the ataxic effects of etomidate.

### EXAMPLE 4

Level of Anaesthesia as Determined by Electroencephalography is Comparable in β2N289S Mice and Wild-Type Mice

Method

In order to compare the depth of anaesthesia in both genotypes, wildtype (n=4) and knock-in (n=4) mice were implanted with radiotelemetry transmitters for the measurement of electroencephalographic (EEG) data. Mice were allowed to recover from the surgical procedure for 14-21 days before experiment. Mice were dosed with 12.5 mg/kg etomidate by the intravenous route and EEG data was continuously sampled. Data sampled at 250 Hz (transmitter bandwidth 0.5-100 Hz, low pass data filter 70 Hz). Data analysed using Somnologica 3 in 100 mSec epochs for % suppression activity per minute. Isoelectric window defined as waveform amplitude less than 5 μV (typical peak to peak amplitude ˜250 μV).

Results

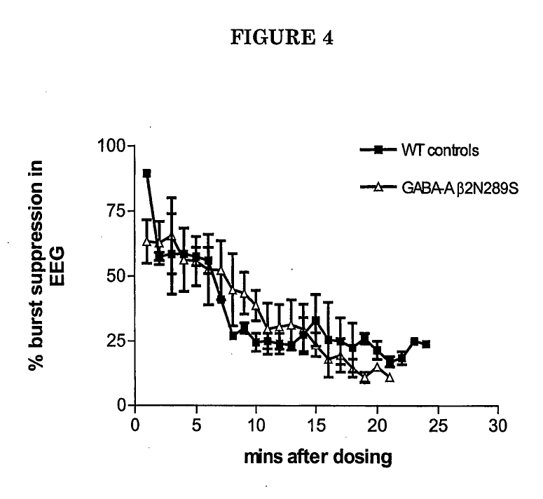

Etomidate anaesthesia results in a characteristic EEG pattern which is called “burst suppression” (Vijn, P. C. M. & Sneyd, J. R., Br. J. Anaes., 81, 415 (1998)). Such activity is characterised by large amplitude, fast spikes interspersed by relatively isoelectric periods in the EEG. Such activity is evident in the EEG of both the wildtype and knock-in mice and quantification of such activity (% of each minute EEG in suppression) reveals no significant difference in the EEG pattern of both genotypes (FIG. 4).

### EXAMPLE 5

Increased Hypnotic Hangover in Wildtype Mice in Comparison to Knock-In Mice Following Etomidate Anaesthesia

Method

In order to quantify the amount of post-anaesthetic sleep, wildtype (n=4) and knock-in (n=4) mice were implanted with radiotelemetry transmitters for the measurement of electroencephalographic (EEG) data. Mice were allowed to recover from the surgical procedure for 13 days before experiment. Baseline EEG data was recorded on day 1 of the experimental protocol. On the 2^{nd} experimental day, mice were dosed with 12.5 mg/kg etomidate by the intravenous route and EEG data was continuously sampled. EEG data sampled at 250 Hz (transmitter bandwidth 0.5-100 Hz, low pass data filter 70 Hz). Data analysed using Somnologica 3 in 8 sec epochs and scored for either “wake” or “slow wave sleep” according to standard electrophysiological criteria (van Gool, W. A. & Mirmiran, M., Sleep, 9, 335 (1986)). Following recovery from etomidate anaesthesia, the amount of slow wave sleep was expressed as percentage of the circadian timed matched baseline.

Results

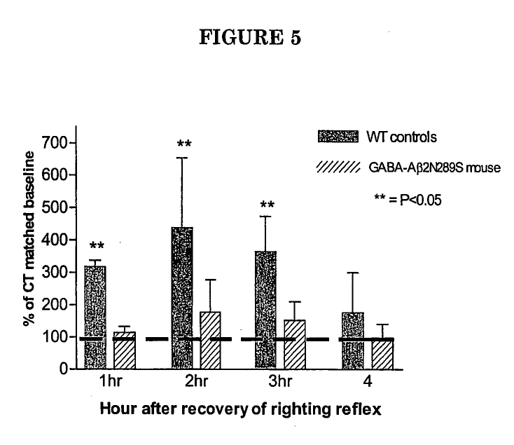

Following etomidate anaesthesia (12.5 mg/kg intravenous route) WT mice have an enhanced level of sleep in comparison to circadian matched baseline data up to 3 hours post recovery (ANOVA P<0.05) whilst the level of slow wave sleep in the knock-in mice following recovery is not statistically different (P>0.05). Wildtype mice therefore have an increased “hypnotic hangover” following recovery from etomidate anaesthesia which may prolong the recovery phase (FIG. 5).

Conclusions

These data suggest that a β₃-selective agonist would still be an anesthetic and would have a reduced potential to impair function following a period of anesthesia.

1-24. (canceled)

25. A transgenic animal whose genomic DNA comprises a GABA_{A} receptor φ₂ subunit gene in which a portion of the open reading frame, being that which encodes the etomidate selectivity site of the GABA_{A} receptor φ₂ subunit polypeptide, has been replaced with a corresponding portion of the GABA_{A} receptor φ₁ subunit open reading frame or a similar manipulation resulting in the same amino acid exchange.

26. The transgenic animal of claim 25 wherein the open reading frame of the GABA_{A} receptor φ₂ subunit is modified such that it encodes a GABA_{A} receptor φ₂ subunit polypeptide with a point mutation at position 289.

27. The transgenic animal of claim 26 wherein the point mutation replaces the endogenous asparagine residue at position 289 of the GABA_{A} receptor φ₂ subunit polypeptide with a serine residue.

28. A transgenic animal whose genomic DNA comprises a gene comprising:
a) an introduced DNA sequence encoding a point mutated GABA_{A} receptor φ₂ subunit polypeptide, the introduced DNA sequence replacing an endogenous sequence encoding at least a portion of a GABA_{A} receptor φ₂ subunit polypeptide; and
b) an endogenous GABA_{A} receptor φ₂ subunit promoter operably linked to the introduced DNA sequence, wherein the transgenic animal exhibits rapid recovery from the anesthetic effects of etomidate, as compared to a reference animal whose genomic DNA does not contain the gene.

29. The transgenic animal of claim 28 wherein the DNA sequence of the GABA_{A} receptor φ₂ subunit is modified such that it encodes a GABA_{A} receptor φ₂ subunit polypeptide with a point mutation at position 289.

30. The transgenic animal of claim 29 wherein the point mutation replaces the endogenous asparagine residue at position 289 of the GABA_{A} receptor φ₂ subunit polypeptide with a serine residue.

31. The transgenic animal of claim 25, wherein the animal is fertile and capable of transmitting the altered GABA_{A} receptor φ₂ subunit gene to its offspring.

32. The transgenic animal of claim 25, wherein said animal is a mouse.

33. A cell line derived from the transgenic animal of claim 25.

34. A method for determining whether a substance is capable of binding to the etomidate binding site of the GABA_{A} receptor φ₃ subunit comprising:
a) exposing the transgenic animal of claims 25 to said substance;
b) determining the response of said transgenic animal to said substance, wherein a change in response compared to the transgenic animal of claims 25, indicates the effect of said compound on GABA_{A} receptor φ₃ subunit activity.

35. The method of claim 34 wherein said response is further compared with the response of a corresponding wildtype animal when exposed to said substance under identical conditions.

36. The method of claim 35 wherein said transgenic animal is a mouse.

37. The method of claim 36 wherein the response to said substance is determined by the rotarod assay.
